(19) United States
(12) Patent Application Publication
(10) Pub. No.: US 2005/0222776 A1
(43) Pub. Date: Oct. 6, 2005

(54) Method for fragment preparation

(75) Inventors: Jennifer L. Ludington, Lansdale, PA (US); Ted T. Fujimoto, Churchville, PA (US); Frank P. Hollinger, Wayne, PA (US)

Correspondence Address: STERNE, KESSLER, GOLDSTEIN & FOX PLLC, 1100 NEW YORK AVENUE, N.W., WASHINGTON, DC, 20005, US

(73) Assignee: Locus Pharmaceuticals, Inc., Blue Bell, PA (US)

(21) Appl. No.: 10/813,553

(22) Filed: Mar. 31, 2004

(51) Int. Cl. G06F 19/00; G01N 33/48; G01N 33/50

(52) U.S. Cl. 702/19; 702/22

(57) ABSTRACT

A method for characterizing a molecular fragment to collect data related to the fragment that allows its evaluation for drug discovery purposes. Starting with a two-dimensional model of the fragment, an initial three-dimensional model of the fragment is derived. Conformers of the fragment are identified. The conformers are then clustered, and a representative conformer is selected from each cluster. An ab initio or semi-empirical calculation and analysis is performed on one or more of the selected conformers. Each atom in the selected conformer is then assigned a type. The selected conformer is analyzed to determine if it is structurally symmetric. If so, the three-dimensional model of the fragment is adjusted to reflect the symmetry. The size of the fragment is calculated to allow geometric analysis of how the fragment physically fits with the protein and/or other fragments. The solvation energy of the fragment is calculated. The free energy curve for the fragment is calculated. Derivatization points for the fragment are then determined; a score is then assigned to each derivatization point, reflecting the ease or difficulty in bonding at the derivatization points. The fragment is then assigned a name and categorized. The fragment and its data derived in the above process can then be stored in a database.

### BACKGROUND OF THE INVENTION

1. Field of the Invention

The invention described herein relates to drug discovery, and in particular relates to the evaluation of candidate molecular fragments.

2. Related Art

Stated broadly, the primary technical issue faced by many pharmaceutical companies is that of discovering or creating one or more molecules that bind to a specific protein in an appropriate manner. In particular, a molecule or molecules must be found that bind to a protein at a specific location, in a particular orientation, and that bind to the protein in a manner that satisfies thermodynamic requirements. One approach for creating such a molecule is to attack the problem at a fragment level. Here, the molecule is engineered one fragment at a time. Any candidate fragments must generally be evaluated one fragment at a time.

To achieve this, a given candidate fragment must be characterized. In particular, the fragment's three-dimensional structure and charge distribution must be determined. In addition, thermodynamic properties must be considered, for example, the solvation energy of the fragment. Moreover, given that the candidate fragment is in fact only a part of what may become a larger molecule, it is necessary to determine where, on the fragment, additional fragments may be attached and how feasible such attachments are.

Currently, there is no method to answer these questions precisely and comprehensively. Therefore, a method is needed to prepare a fragment, i.e., collect data related to a candidate fragment that facilitates the evaluation of that fragment as a possible building block of a larger molecule.

### SUMMARY OF THE INVENTION

The invention described herein is a method for characterizing a molecular fragment so as to collect data related to the fragment. This data allows evaluation of the fragment for drug discovery purposes. Starting with a two-dimensional model of the candidate fragment, an initial three-dimensional model of the fragment is derived. Conformers of the fragment are identified. The conformers are then grouped, or clustered, and a representative conformer is selected from each cluster. An ab initio or semi-empirical electronic calculation is then performed on one or more of these selected conformers to characterize the geometry and charge distribution of the conformer. Each atom in a selected conformer is assigned a category, or type. The selected conformer is analyzed to determine if it is structurally symmetric. If so, the three-dimensional model of the fragment is adjusted to reflect the symmetry. The size of the fragment is calculated to allow analysis as to how the fragment physically fits with the protein and/or other fragments. The solvation energy of the fragment is also calculated. The free energy curve for the fragment is calculated. Derivatization points for the fragment are determined; a score is then assigned to each derivatization point, reflecting the ease or difficulty in bonding at the derivatization points. The fragment is assigned a name and categorized. The candidate fragment and its characterizing data derived in the above process can then be stored in a database.

Further embodiments, features, and advantages of the present invention, as well as the operation of the various embodiments of the present invention, are described below with reference to the accompanying drawings.

### BRIEF DESCRIPTION OF THE FIGURES

FIG. 1 is a flowchart illustrating the context of the invention in a drug discovery process.

FIGS. 2A and 2B illustrate the steps of an embodiment of the invention.

FIG. 3 is a flowchart illustrating the steps of deriving an initial three-dimensional model of a fragment, according to an embodiment of the invention.

FIG. 4 is a flowchart illustrating the steps of deriving conformations of a fragment, according to an embodiment of the invention.

FIG. 5 is a flowchart illustrating the steps of executing an ab initio or semi-empirical calculation on one or more of these selected conformers to characterize the geometry and charge distribution of the conformer, according to an embodiment of the invention.

FIG. 6 is a flowchart illustrating the steps of determining the type of a particular atom of a fragment, according to an embodiment of the invention.

FIGS. 7A, 7B and 7C illustrate the steps of symmetrizing a fragment, according to an embodiment of the invention.

FIGS. 8A and 8B illustrate two molecular structures and their respective symmetries.

FIG. 9 is a flowchart illustrating the steps of calculating a fragment-fragment cutoff, according to an embodiment of the invention.

FIG. 10 is an exemplary free energy curve as determined by the processing of the invention, according to an embodiment of the invention.

FIG. 11 is a flowchart that illustrates the steps of calculating a an energy offset for purposes of developing a self-association free energy curve for the fragment, according to an embodiment of the invention.

FIG. 12 is a flowchart that illustrates the steps of determining derivatization points of a fragment and assigning a score to each derivatization point, according to an embodiment of the invention.

FIG. 13 is a block diagram illustrating a computing platform for a software implementation of the invention.

### DETAILED DESCRIPTION OF THE INVENTION

Embodiments of the present invention are now described with reference to the figures, where like reference numbers indicate identical or functionally similar elements. Also in the figures, the leftmost digit of each reference number corresponds to the figure in which the reference number is first used.

While specific configurations and arrangements are discussed, it should be understood that this is done for illustrative purposes only. A person skilled in the relevant art will recognize that other configurations and arrangements can be used without departing from the spirit and scope of the invention. It will be apparent to a person skilled in the relevant art that this invention can also be employed in a variety of other systems and applications.

I. Overview

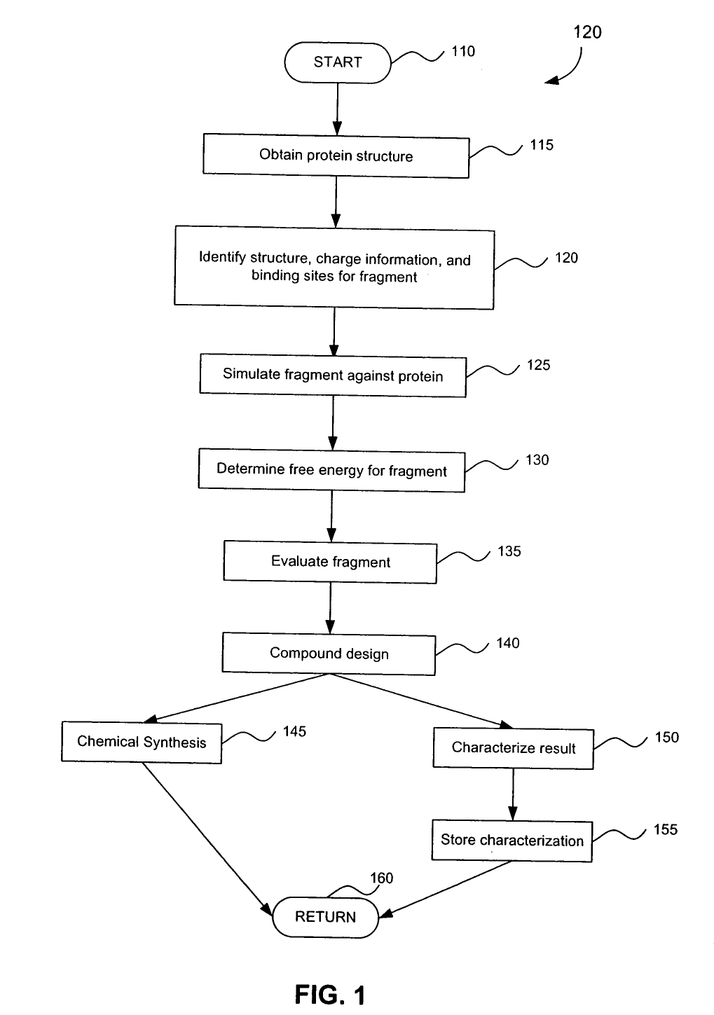

The invention described herein represents a method for obtaining information about a fragment, wherein the information allows subsequent evaluation of the fragment as candidate for use in creating a drug. FIG. 1 illustrates a process by which a candidate fragment can be analyzed, evaluated, and used in the design of a such a drug. The process begins at step 110. In step 115, the structure of a target protein is obtained. This is the protein with respect to which a given fragment is to be analyzed and evaluated. Of interest here is whether a given fragment will bind to the protein at an appropriate location, such that the necessary thermodynamic requirements are met in the binding process. Moreover, a fragment that is ultimately chosen must have the appropriate structure and charge characteristics. The protein structure obtained in step 115 can be a computer representation of the protein. Such a computer representation can be obtained from a publicly accessible database. The protein structure may have been derived using x-ray crystallography, or other means. Typically, the representation of the protein includes a three-dimensional structure that specifies the positions of particular atoms and the bonds between them.

In step 120, the structure of a candidate fragment is determined, along with information regarding the charges at various points in the structure, and derivatization points of the fragment at which other fragments can be attached.

In step 125, the interaction between the fragment and the protein is simulated. Conceptually, this simulation can entail the analysis of a system comprising an instance of the protein and numerous instances of the fragment. An evaporation process is then simulated, such that fragments that have not bound to the protein are evaporated or otherwise lost from the system. After a phase transition, what remains are fragments “bound” to the protein. This serves to reveal particular binding sites on the protein. Moreover, it is also necessary to determine the free energy for the fragment with respect to the protein. This determination is made is step 130, which is discussed in greater detail in U.S. patent application Ser. 10/784,708, filed Dec. 31, 2003, and incorporated herein by reference in its entirety.

Given that information has been collected regarding the fragment in relation to the protein in the above steps, in step 135 an evaluation of the fragment is performed. This represents a determination as to whether to proceed with the fragment to the synthesis stage. If the evaluation is favorable, the process continues at step 140. Here, a molecule can be engineered incorporating the evaluated fragment. Step 140 includes, for example, determination of the appropriate bond angles and lengths, as well as the necessary torsions in the molecular structure. Step 140 further provides information as to whether actual synthesis of the molecule is practical.

If so, the molecule may actually be synthesized in step 145. Independent of whether or not the molecule is synthesized, the information gained from the above steps can be compiled and organized for future reference. This takes place in step 150. This compilation of the results of the preceding analysis represents a characterization of the fragment. This characterization can then be stored in step 155. This characterization can be stored electronically, for example, in a database format using a commercially available database package. The process concludes at step 160.

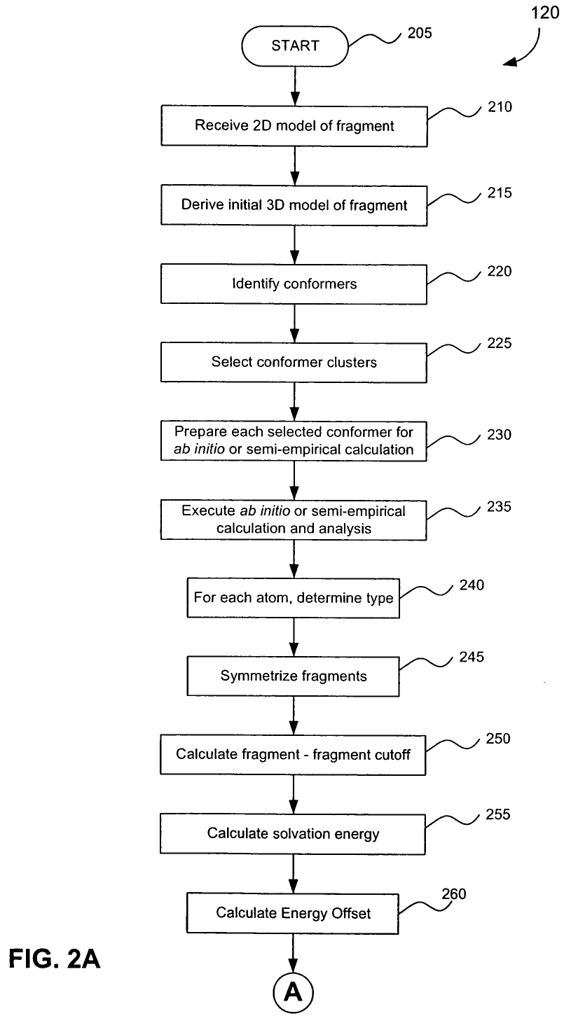

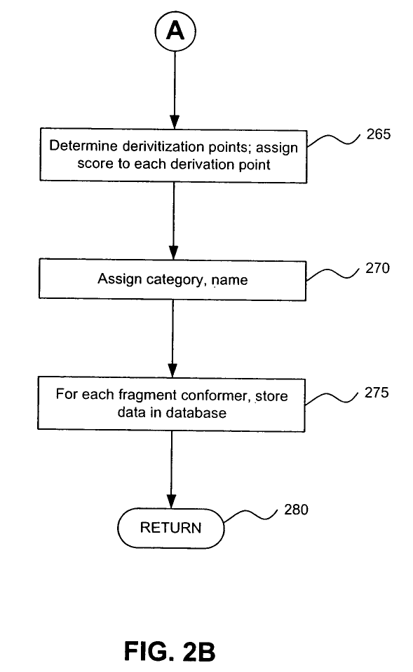

An embodiment of fragment preparation, step 120 above, is illustrated generally in FIGS. 2A and 2B. The process begins at step 205. In step 210, a two-dimensional model of the fragment is received. In step 215, a three-dimensional model of the fragment is derived from this two-dimensional model. In step 220, relevant structural conformers of the fragment are identified. In step 225, the conformers are organized into clusters on the basis of similarity. For each cluster, a single conformer is selected as a representative of the cluster. In step 230, each selected conformer is prepared for an ab initio or semi-empirical calculation. In step 235, the ab initio or semi-empirical calculation is executed.

In step 240, each atom of a given conformer is assigned to a particular type that is based on a variety of factors, including the element of the atom, its bonds, and the structures to which the atom is bonded. In step 245, the conformer is symmetrized. Here, a determination is made as to whether a fragment should be symmetrical, given its known molecular structure. If so, a determination is made as to whether corresponding bond lengths (i.e., those lengths that should be equal if symmetry is presumed) are in fact equal in the existing model of the fragment as derived in the above steps. If not, the corresponding bond lengths of the fragment model are adjusted so as to achieve this presumed symmetry. Likewise, a determination is made as to whether corresponding bond angles are equal in the existing model. If not, the bond angles of the fragment model are adjusted to achieve this presumed symmetry.

In step 250, the size of a fragment is calculated for purposes of geometric analysis. A measure of the size of the fragment is denoted here as the fragment-fragment cutoff. This provides information that allows analysis of whether a particular fragment will fit in a particular location, in light of the topologies of the protein and/or other neighboring fragments. The fragment-fragment cutoff can also be used by an energy evaluation algorithm as a measure of when to include or exclude a fragment or atoms of the fragment in an energy evaluation step. In step 255, the solvation energy of the fragment is calculated. In step 260, the B-shift for the fragment is calculated. As will be described in greater detail below, this allows for expedited computation of the free energy curve of the fragment.

In step 265, the derivatization points of the fragment are determined, and a score is assigned to each derivatization point. The score indicates the ease or difficulty of bonding another structure to that derivatization point. In step 270, the fragment is assigned to a category and assigned a particular name. In step 275, all the information derived above for the given fragment conformer is stored. Such information can be stored electronically in a database, for example. The process concludes at step 280.

II. Processing, Fragment Preparation

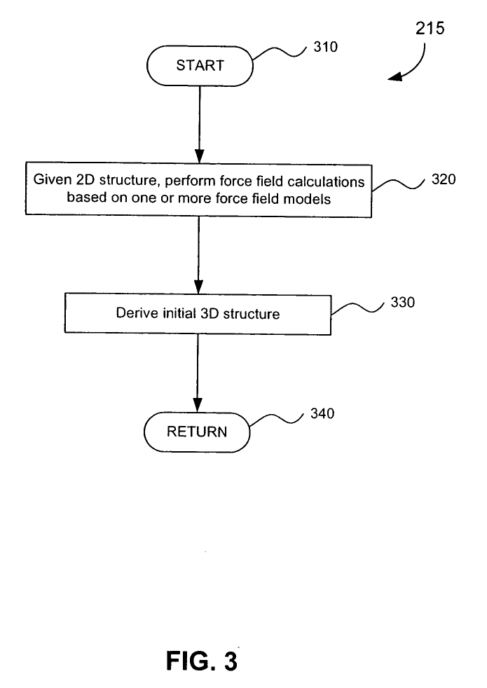

As described above, in particular embodiments, the first step in the fragment preparation process is to receive a two-dimensional model of a fragment. The next step is to derive an initial three-dimensional model of the fragment on the basis of the received two-dimensional model. This derivation is illustrated in more detail in FIG. 3. The process begins at step 310. In step 320, force field calculations are performed, given the two-dimensional structure, based on one or more force field models. Here, a molecular mechanics approach is used in developing the three-dimensional structure. As would be known to one of skill in the art, any of several force field models can be used, alone or in combination. These include the AMBER model (Kollman), the OPLS model (Jorgensen), the MMX model (Allinger), and the Merck Molecular Force Field (MMFF) model (Halgren). In step 330, an initial three-dimensional structure is derived on the basis of the force field calculations of step 320. The process concludes at step 340.

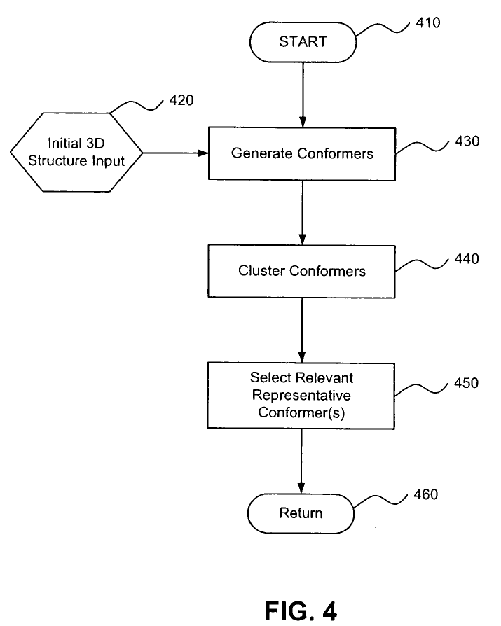

Once an initial three-dimensional model of the fragment is constructed, conformers of the fragment can be identified which begins with step 410 of FIG. 4. Clearly, this identification is not necessary if the fragment only has one conformer. If, however, a fragment has more than one conformer, all relevant conformers should be identified, analyzed, and evaluated to some extent in step 430. In order to streamline the fragment preparation process, however, given a plurality of conformers, the set of conformers of a fragment can be grouped into clusters according to their structural similarity as is done in step 440. For each cluster in step 450, one conformer can be selected as a representative of the cluster. Analysis and evaluation can then proceed with respect to the selected conformers. In this way, the process can continue without having to analyze and evaluate every individual conformer in detail.

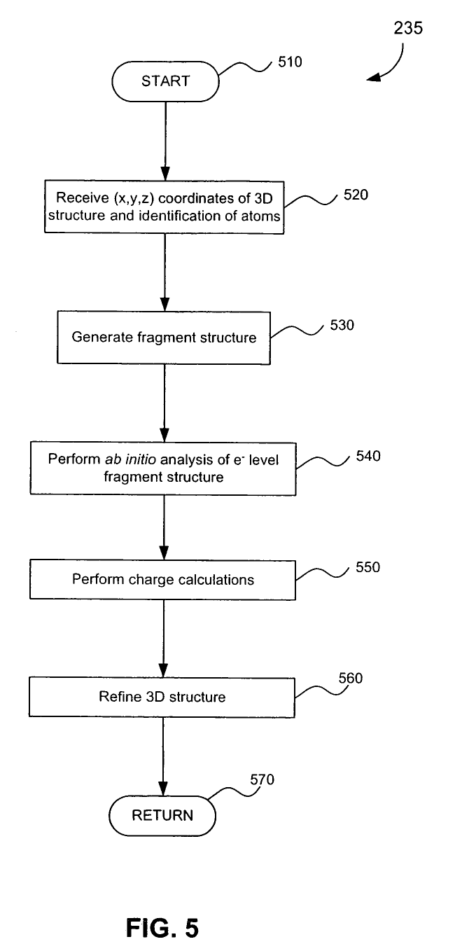

A selected conformer can then be prepared for an ab initio or semi-empirical calculation. The ab initio or semi-empirical calculation and analysis is illustrated in greater detail in FIG. 5. Generally, this process takes an ab initio approach to further refine the three-dimensional model of the fragment. The process starts in step 510. In step 520, the (x, y, z) coordinates of the three-dimensional structure are received, along with identification of each particular atom in the fragment. In step 530, the structure of the fragment is determined at the electron (e-) level. In step 540, the ab initio analysis of the electron level fragment structure is performed. In step 550, charge calculations are performed, and in step 560, the three-dimensional structure is refined. The process concludes at step 570.

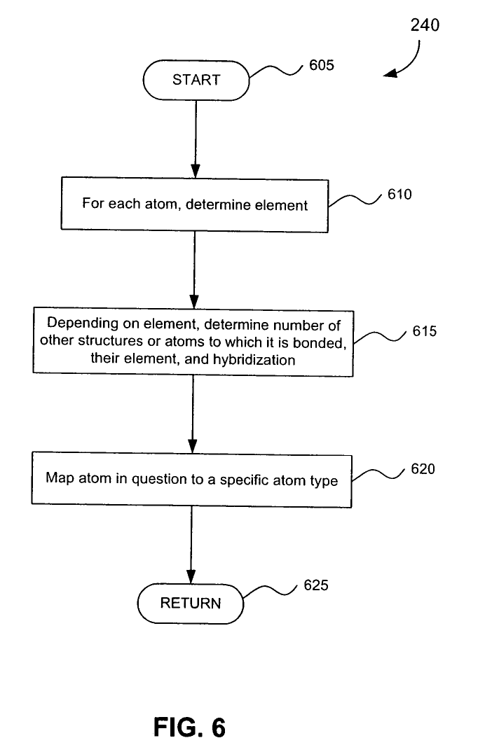

Each atom in the fragment under analysis is then assigned to a particular atom type. The process for this classification is illustrated in greater detail in FIG. 6. The process begins with step 605. In step 610, for each atom, its element is determined. In step 615, depending on the element, additional information about the atom is determined. This additional information can include, for example, the number of other atoms or structures to which the atom is bonded, the element of those other atoms, and the hybridization involved in bonding to those atoms or structures. In step 620, the atom in question is mapped to a particular type. The process concludes at step 625.

One scheme under which atoms can be typed is illustrated in the following table. For each element, the type's name is given, followed by the definition of the type.

Carbon:

| | |
|---|---|
| CT | Bonded to 4 other atoms |
| C | Bonded to 3 other atoms |
| CZ | Bonded to 2 other atoms |

Nitrogen:

| | |
|---|---|
| N3 | Attached to 4 other atoms (formal positive charge) |
| NT | Attached to 3 other atoms and not sp2 |
| N | Attached to 2 other atoms |
| | or |
| | Attached to 3 other atoms and |
| | in an aromatic ring or |
| | attached to an aromatic ring or |
| | is an amide nitrogen |
| NY | Attached to 1 other atom |

Oxygen:

| | |
|---|---|
| OH | Attached to 2 atoms, one of which is hydrogen |
| OS | Attached to 2 atoms, which are non-hydrogen |
| O | Attached to 1 atom |

Phosphorus:

| | |
|---|---|
| P | Any phosphorous |

Sulphur:

| | |
|---|---|
| SH | Attached to 1 hydrogen |
| S | Any sulfur not bonded to hydrogen |

Hydrogen:

| | |
|---|---|
| H | Attached to nitrogen |
| HS | Attached to sulfur |
| HO | Attached to TO oxygen |
| HP | Attached to a carbon bonded to a positively charged N (N3) |
| HC | Attached to aliphatic carbon with 0 electron withdrawing |
| | substituents (EWS) |
| H1 | Attached to aliphatic carbon with 1 EWS |
| H2 | Attached to aliphatic carbon with 2 EWS |
| H3 | Attached to aliphatic carbon with 3 EWS |
| HA | Attached to aromatic carbon with 0 electronegative neighbors |
| | (ENN) |
| H4 | Attached to aromatic carbon with 1 ENN |
| H5 | Attached to aromatic carbon with 2 ENN |

Halogens:

| | |
|---|---|
| F | Any fluorine |
| Cl | Any chlorine |
| Br | Any bromine |
| I | Any iodine |

Atoms not fitting any of these categories can be flagged for later analysis.

The above chart is meant to be exemplary only; other classification schemes can also be used in addition to or in conjunction with the above scheme without departing from the spirit or scope of the invention.

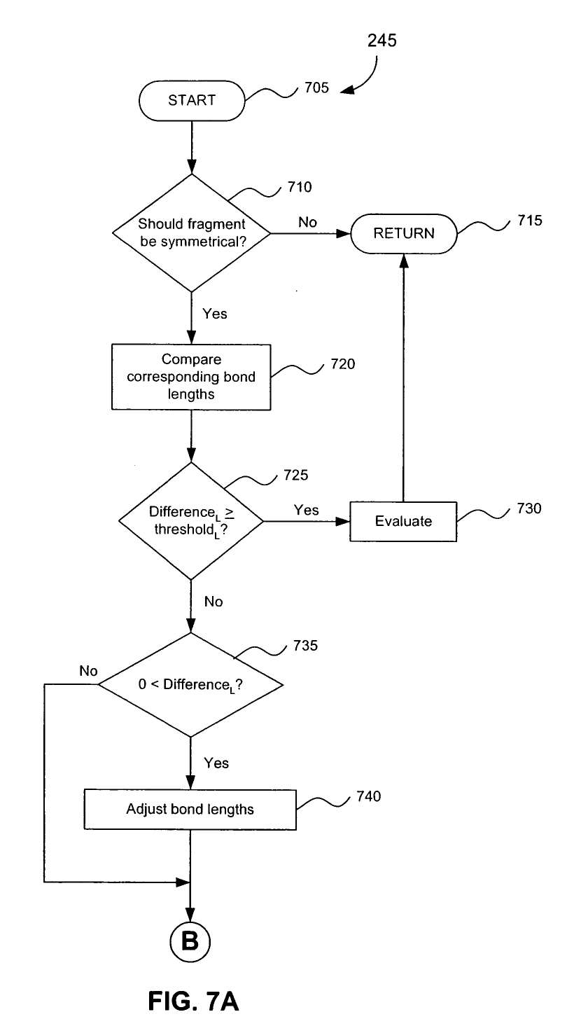

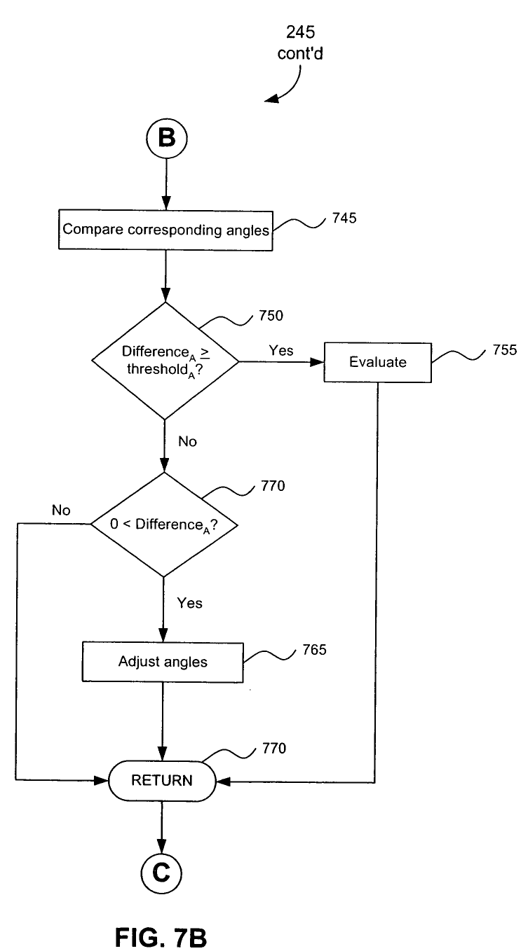

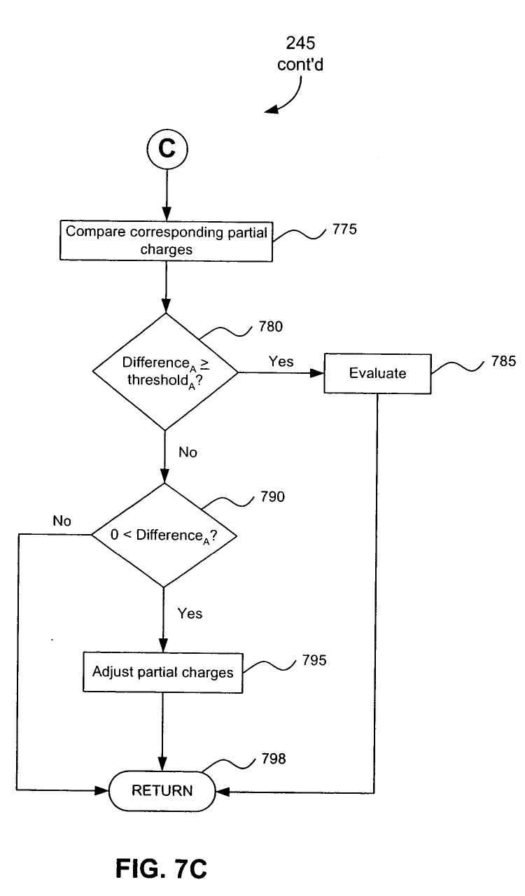

At this point, a three-dimensional model of the fragment has been derived and refined. Some fragments can be further refined with respect to their structural model by determining whether or not the fragment should be symmetric. If so, the bond lengths; bond angles and partial charges of the atoms of the three-dimensional model can be adjusted to achieve symmetry. This process is illustrated in greater detail in FIGS. 7A, 7B and 7C. The process begins at step 705. In step 710, a determination is made as to whether the fragment should be symmetrical. If not, then there is no point in verifying the symmetry of the current three-dimensional model. The process would then conclude at step 715. If it is determined that the fragment is symmetrical, then the process continues at step 720. Here, corresponding bond lengths (i.e., the lengths of bonds that should be equal, given the symmetry) are compared. In step 725, a determination is made as to whether the difference in bond lengths exceeds some threshold value. In the illustration, the difference is denoted “difference_{L}” while the threshold is denoted “threshold_{L}.” Threshold_{L} is a predetermined value which, if exceeded by difference_{L}, indicates that asymmetry exists in the current three-dimensional model. If such a symmetry is present, then the current model can be evaluated offline in step 730. If, however, difference_{L} is less than threshold_{L}, then the process continues at step 735. Here, a determination is made as to whether difference_{L} is greater than zero. If so, then the corresponding bond lengths are adjusted in step 740. In an embodiment of the invention, the bond lengths can be averaged. The average bond length can then be substituted for each of the corresponding bond lengths. If, however, difference_{L} does not exceed zero, then there is no point in adjusting bond lengths. The process then continues at step 745.

At step 745, corresponding bond angles are compared. In step 750, a determination is made as to whether the difference between two corresponding bond angles exceeds a predetermined threshold value. Here, the difference in bond angles is referred to as “difference_{A}”, while the threshold is denoted “threshold_{A}.” Again, if difference_{A} exceeds threshold_{A}, then significant asymmetry is present, and the fragment can be evaluated offline in step 755. Otherwise, the process continues at step 760. Here, a determination is made as to whether threshold_{A} exceeds zero. If so, then the process continues at step 765, where the corresponding bond angles are adjusted. In an embodiment of the invention, corresponding bond angles are adjusted by averaging. The average bond angle is then substituted for each of the corresponding angles. If threshold_{A} does not exceed zero in step 760, then there is no need to adjust the bond angles and the process of comparing bond angles is concluded at step 770.

At step 775, corresponding partial charges are compared. In step 780, a determination is made as to whether the difference between two corresponding partial charges exceeds a predetermined threshold value. Here, the difference in partial charges is referred to as “difference_{A}”, while the threshold is denoted “threshold_{A}.” Again, if difference_{A} exceeds threshold_{A}, then significant asymmetry is present, and the fragment can be evaluated offline in step 785. Otherwise, the process continues at step 790. Here, a determination is made as to whether threshold_{A} exceeds zero. If so, then the process continues at step 795, where the corresponding partial charges are adjusted. If threshold_{A} does not exceed zero in step 790, then there is no need to adjust the partial charges and the process concludes at step 798.

In an embodiment of the invention, the partial charges can be averaged. The average partial charge can then be substituted for each of the corresponding partial charges. If, however, difference_{L} does not exceed zero, then there is no point in adjusting the partial charges.

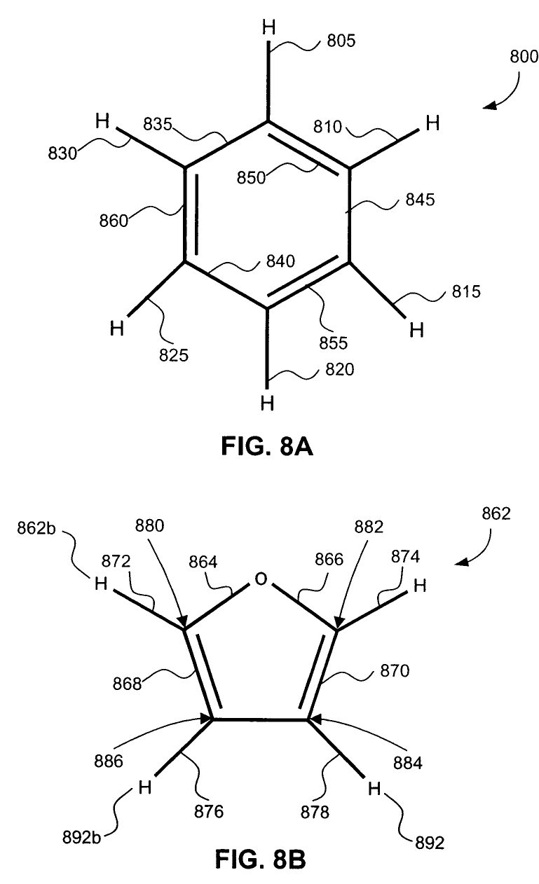

An example of a symmetrical fragment is illustrated in FIG. 8A. Here, the lengths of bonds 805, 810, 815, 820, 825, and 830 should all be equal, since these bonds correspond to one another. If, however, the existing three-dimensional model of this fragment shows that these bond lengths are not equal, then the process described above with respect to FIGS. 7A, 7B and 7C is performed. Likewise, the lengths of bonds 835, 840, and 845 should be equal. Similarly, the lengths of bonds 850, 855, and 860 should be equal.

Another symmetrical molecule is illustrated in FIG. 8B. Here, the lengths of bonds 864 and 866 should be equal. Similarly, the lengths of bonds 868 and 870 should be equal. Bonds 864 and 866 represent corresponding bonds, as do bonds 868 and bonds 870. Also, bonds 872 and 874 represent corresponding bonds, such that their lengths should be equal. Similarly, bonds 876 and 878 should be equal in length. If any of these corresponding bond lengths are not equal, then it is appropriate to execute the process illustrated in FIGS. 7A and 7B. In addition, angles 880 and 882 represent corresponding bond angles. These two bond angles are compared in the process of FIGS. 7A and 7B, such that if they are not equal, they would be adjusted, assuming that their difference is not substantial. Likewise, bond angles 886 and 884 represent corresponding bond angles.

Another determination that can be made in this invention is the fragment-fragment cutoff. The fragment-fragment cutoff represents the size of a fragment. This size is used as a unit of distance for analytical purposes. If a fragment is a certain number of units away from another fragment, the interaction between the two fragments can be ignored for modeling purposes. Also, fragments may attach themselves to a protein in layers. Any fragment that is outside the innermost layer of fragments (i.e., outside the monolayer) can be disregarded for modeling purposes. It is the fragments that are in the monolayer that might represent fragments of interest. The monolayer of fragments can be characterized by considering the distance of such a fragment from the protein, as measured by the fragment-fragment cutoff distance.

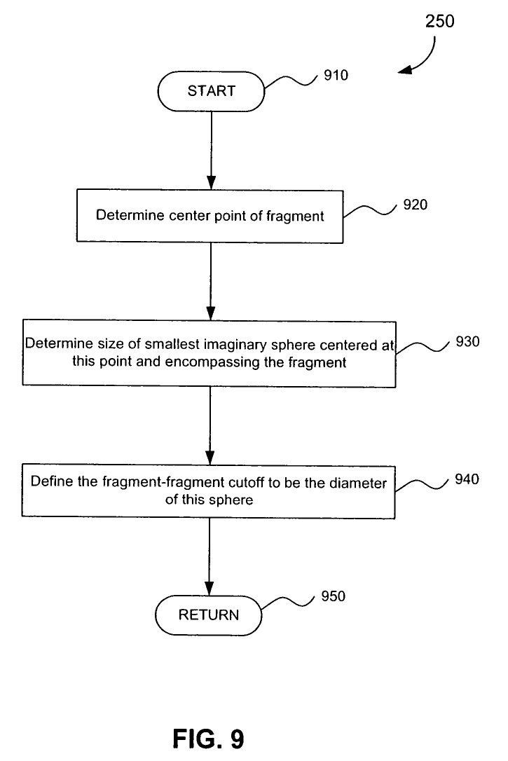

The determination of a fragment-fragment cutoff is illustrated in greater detail in FIG. 9. The process begins at step 910. In step 920, the center point of a fragment is determined. The center point can be determined by considering the physical structure of the fragment (i.e., a geometric approach). In an alternative embodiment of the invention, the center point of a fragment can be defined as the center of mass of the fragment. In step 930, an imaginary sphere is created, wherein the sphere is centered at the center point of the fragment. The sphere is made large enough to encompass the fragment, but no larger. Hence the process determines the size of the smallest imaginary sphere that is centered at the center point of the fragment. In step 940, the fragment-fragment cutoff is defined to be the diameter of this sphere. The process concludes at step 950.

In addition to the fragment-fragment cutoff, it is also useful to calculate the solvation energy of a fragment. Conceptually, the solvation energy for a fragment refers to the energy required to break its interaction with a solvent, along with any energy recovered if and when the fragment bonds to the protein. Generally, there are several ways to calculate solvation energy. One is the use of a continuum solvent model. One example is the general born/surface area (GB/SA) model. This model is often used for small fragment molecules to calculate the free energy of solvation. Another method is to use MacroModel (Maestro), a commercially available product (Schrödinger, LLC, Portland, Oreg.). Other models that can be used to calculate solvation energy include the TIP3P, TIP4P, TIP4P models and the Poisson-Boltzmann model.

The invention also includes a process for generating a free energy curve for a fragment. The process of simulating a fragment against a given protein can be viewed conceptually as a system that includes an instance of the protein molecule and a plurality of instances of a fragment. In the simulation, free fragments are allowed to leave the system, lowering the total number of fragments in the system. Eventually, the system has a lower energy ΔG, given that free fragments have left the system in a process akin to evaporation. Remaining in the system at this point would be the protein molecule along with whatever fragments have bonded to the protein. A free energy curve represents the change in the number of fragments in the system as the free energy decreases, given the loss of the free fragments.

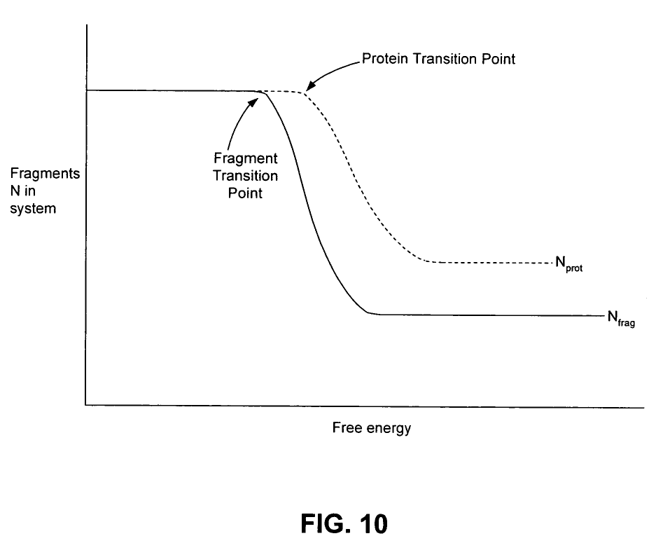

A free energy curve is illustrated in FIG. 10. The free energy for a system such as the one just described is shown as curve Nₚᵣₒₜ. Generally, the number of fragments in the system decreases as free energy in the system decreases.

Also shown in FIG. 10 is a second curve, N_{frag}. This curve represents the fragment-fragment interaction free energy in a system containing only fragments, without a protein molecule. In determining a fragment-fragment energy curve, Nₚᵣₒₜ, the curve N_{frag} represents a limiting case. In particular, the transition point of the curve N_{frag} will always precede the transition point of the curve Nₚᵣₒₜ. This is because in the case of N_{frag}, there is no protein present in the system. Therefore, given that there is no protein to which a fragment can interact, fragments will disassociate from one another until there are no fragments remaining in the system. Contrasting this with a system which also contains a protein for the fragments to interact, the fragments will first dissociate from one another (the point approximately at N_{frag}) then will begin to be removed from the protein surface, Nₚᵣₒₜ and beyond.

An energy offset can be calculated from the N_{frag} curve which can aid in determining the free energy schedule of the simulation between the protein and the fragment. The energy offset aids in determining when the transition point for the protein-fragment free energy curve is approaching. Accordingly, calculating and using the free energy offset in the protein-fragment simulation saves computer time by allowing the free energy to change in relatively large increments prior to the energy offset.

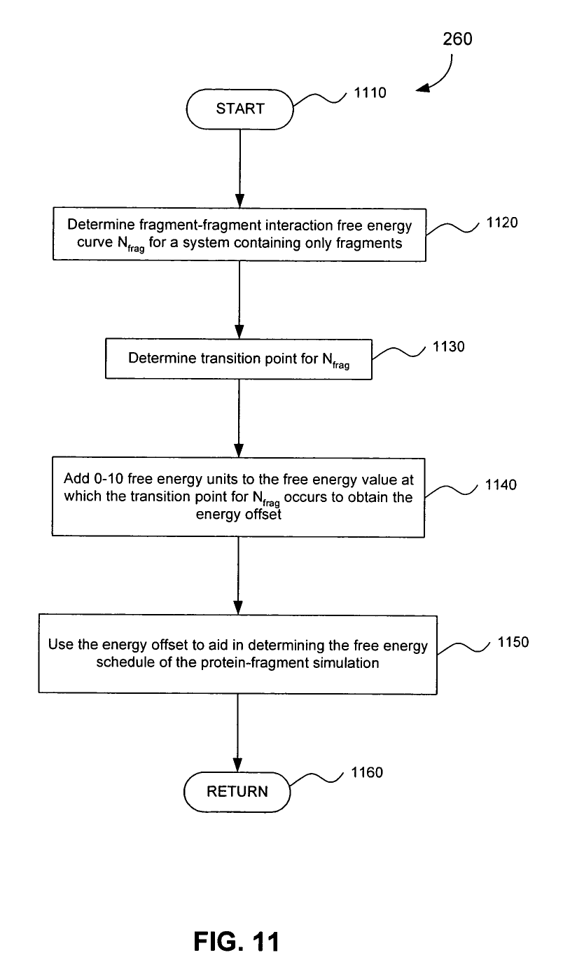

FIG. 11 demonstrates an embodiment of how the energy offset is determined. At step 1120 a fragment-fragment interaction free energy curve N_{frag} is calculated for system containing only fragments. At step 1130 the transition point for N_{frag} is determined. At step 1140, a particular number of free energy units is added to the free energy value at which the transition point for N_{frag} occurs to obtain the energy offset. In particular embodiments, between 0 and 10 free energy units are added to the free energy value of the fragment-only transition to arrive at the energy offset. At step 1150, the energy offset value is then used to aid in determining the free energy schedule of the protein-fragment simulation.

In the protein-fragment simulation, prior to the energy offset, the free energy is changed in relatively large increments. As the free energy in the protein-fragment simulation approaches the energy offset, the increments at which the free energy changes become smaller. The ability to change the free energy in relatively large increments prior to the energy offset saves valuable computational time.

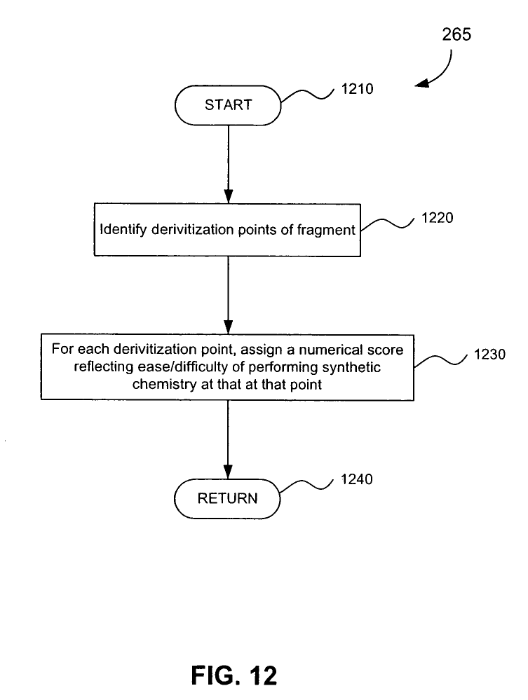

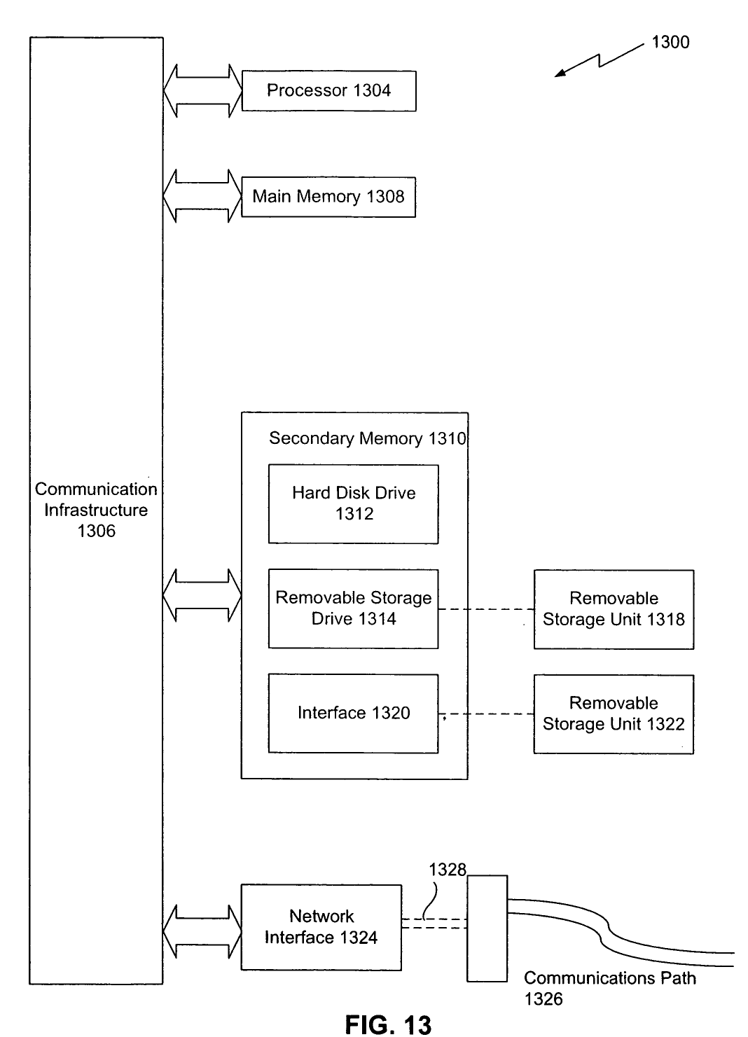

It is also useful to determine the derivatization points on a fragment. A derivatization point represents a point on a fragment where additional atoms or structures can be bonded. This information is useful for purposes of determining what molecules can be generated by building on the fragment. Moreover, it is also useful to determine how easy or difficult it is to synthesize or modify a molecule at a given derivatization point. This process is illustrated in FIG. 12. The process begins at step 1210. In step 1220, derivatization points of a fragment are identified. In step 1230, for each derivatization point, a numerical score is assigned. The score reflects the ease or difficulty of bonding at that point. The process concludes at step 1240. This process allows a ready determination as to whether a fragment can be used for constructing other, larger molecules, and provides information on how easy or difficult such a synthesis would be. Note that the scores can be based on pre-existing knowledge. Moreover, the scoring allows a ranking of fragments according to synthetic feasibility.

Once all the above has been performed, it can be useful to assign a name to the fragment and/or assign the fragment to a category. The name can be that used by the International Union of Pure and Applied Chemists (IUPAC) or the common name. Generally, the name is unique for every conformer.

Categorization of the fragments can be performed for purposes of organization of data accumulated with respect to a given protein. There are a number of categories which can be used. For example, a fragment can be categorized as a scaffold. This indicates that the fragment can be used as a frame on which a larger molecule can be constructed. A fragment can also be categorized as a linker, indicating that the fragment can be used to link two or more other molecular structures. Alternatively, a fragment can be categorized as a hydrophobe, a hydrogen bond acceptor, or a hydrogen bond donor. Note that these categories are not mutually exclusive. In yet a third scheme, categories can be substructure based. A fragment can, for example, be a benzene core molecule, a biphenyl core molecule, or a diphenyl ether core.

Finally, all of the above information can be stored in a database. Existing, commercially-available databases can be used for this process. The stored information can include, for example, one-, two-, or three-dimensional structural information as derived above.

III. Computing Environment

Some or all of the present invention may be implemented using software and may be implemented in conjunction with a computing system or other processing system. An example of such a computer system 1200 is shown in FIG. 12. The computer system 1200 includes one or more processors, such as processor 1204. The processor 1204 is connected to a communication infrastructure 1206, such as a bus or network. Various software implementations are described in terms of this exemplary computer system. After reading this description, it will become apparent to a person skilled in the relevant art how to implement the invention using other computer systems and/or computer architectures.

Computer system 1200 also includes a main memory 1208, preferably random access memory (RAM), and may also include a secondary memory 1210. The secondary memory 1210 may include, for example, a hard disk drive 1212 and/or a removable storage drive 1214, representing a magnetic tape drive, an optical disk drive, etc. The removable storage drive 1214 reads from and/or writes to a removable storage unit 1218 in a well-known manner. Removable storage unit 1218 represents a magnetic tape, optical disk, or other storage medium that is read by and written to by removable storage drive 1214. As will be appreciated, the removable storage unit 1218 can include a computer usable storage medium having stored therein computer software and/or data.

In alternative implementations, secondary memory 1210 may include other means for allowing computer programs or other instructions to be loaded into computer system 1200. Such means may include, for example, a removable storage unit 1222 and an interface 1220. An example of such means may include a removable memory chip (such as an EPROM, or PROM) and associated socket, or other removable storage units 1222 and interfaces 1220 which allow software and data to be transferred from the removable storage unit 1222 to computer system 1200.

Computer system 1200 may also include one or more communications interfaces, such as network interface 1224. Network interface 1224 allows software and data to be transferred between computer system 1200 and external devices. Examples of network interface 1224 may include a modem, a network interface (such as an Ethernet card), a communications port, a PCMCIA slot and card, etc. Software and data transferred via network interface 1224 are in the form of signals 1228 which may be electronic, electromagnetic, optical or other signals capable of being received by network interface 1224. These signals 1228 are provided to network interface 1224 via a communications path (i.e., channel) 1226. This channel 1226 carries signals 1228 and may be implemented using wire or cable, fiber optics, an RF link and other communications channels.

In this document, the terms “computer program medium” and “computer usable medium” are used to generally refer to media such as removable storage units 1218 and 1222, a hard disk installed in hard disk drive 1212, and signals 1228. These computer program products are means for providing software to computer system 1200.

Computer programs (also called computer control logic) are stored in main memory 1208 and/or secondary memory 1210. Computer programs may also be received via communications interface 1224. Such computer programs, when executed, enable the computer system 1200 to implement the present invention as discussed herein. In particular, the computer programs, when executed, enable the processor 1204 to implement the present invention. Accordingly, such computer programs represent controllers of the computer system 1200. Where the invention is implemented using software, the software may be stored in a computer program product and loaded into computer system 1200 using removable storage drive 1214, hard drive 1212 or communications interface 1224.

IV. Conclusion

While various embodiments of the present invention have been described above, it should be understood that they have been presented by way of example, and not limitation. It will be apparent to persons skilled in the relevant art that various changes in detail can be made therein without departing from the spirit and scope of the invention. Thus the present invention should not be limited by any of the above-described exemplary embodiments.

1. A method of deriving data regarding a fragment molecule that is under evaluation regarding its interaction with a protein, the method comprising:
(a) identifying conformers of the fragment;
(b) selecting conformers that are representative of clusters of the identified conformers; and
(c) performing the ab initio or semi-empirical calculation and analysis on the selected conformer.

2. The method of claim 1, further comprising:
(a) for each atom of the selected conformer, determining an atom type.

3. The method of claim 1, further comprising:
(a) symmetrizing the selected conformer if symmetry is recognized.

4. The method of claim 1, further comprising:
(a) calculating a fragment-fragment cutoff for the selected conformer.

5. The method of claim 1, further comprising:
(a) calculating a solvation energy for the selected conformer.

6. The method of claim 1, further comprising:
(a) calculating an energy offset for the selected conformer.

7. The method of claim 1, further comprising:
(a) determining a derivatization point of the selected conformer; and
(b) assigning a score to the derivatization point indicative of the ease of bonding at the derivatization point.

8. The method of claim 1, further comprising:
(a) assigning the selected conformer to a category.

9. The method of claim 1, further comprising:
(a) assigning a name to the selected conformer.

10. The method of claim 1, further comprising:
(a) storing data derived in steps (a) through (m) in a database.

11. The method of claim 1, wherein said step (b) comprises performing force field calculations based on one or more force field models.

12. The method of claim 1, wherein said step (c) comprises performing conformational searches of the fragment and identifying relevant conformations of a fragment.

13. The method of claim 1, wherein said step (d) comprises performing cluster analysis of the relevant conformations identified in step (6) and selecting the representative conformation or conformations in the identified clusters.

14. The method of claim 1, wherein said step (f) comprises:
(i) receiving (x,y,z) coordinates of the initial three-dimensional model of the selected conformer;
(ii) determining structure of the selected conformer at an electron level of detail;
(iii) performing ab initio analysis of the electron-level structure of the selected conformer;
(iv) performing charge calculations on the electron-level structure; and
(v) refining the initial three-dimensional model on the basis of the ab initio analysis and the charge calculations.

15. The method of claim 1, wherein said step (g) comprises:
(i) determining the element of the atom; and
(ii) mapping the atom to a type based on the element, any structures to which the atom is bonded, and hybridization.

16. The method of claim 1, wherein said step (h) comprises:
(i) recognizing if symmetry is present in the selected conformer;
(ii) if so, comparing corresponding bond lengths; and
(iii) if a difference is found in the corresponding bond lengths, and if the difference is below a threshold value, adjusting the corresponding bond lengths.

17. The method of claim 16, wherein said step (iii) comprises:
averaging the corresponding bond lengths to produce an average bond length; and
replacing each corresponding bond length with the average bond length.

18. The method of claim 1, wherein said step (h) comprises:
(i) recognizing if symmetry is present in the selected conformer;
(ii) if so, comparing a pair of corresponding bond angles; and
(iii) if a difference is found in bond angles, and if the difference is above a threshold value, adjusting the corresponding bond angles.

19. The method of claim 18, wherein said step (iii) comprises:
averaging the corresponding bond angles to produce an average bond angle, and
replacing each corresponding bond angle with the average bond angle.

20. The method of claim 1, wherein said step (h) comprises:
(i) Recognizing if symmetry is present in the selected conformer;
(ii) If so, comparing a pair of corresponding partial charges on symmetrical atoms; and
(iii) if a difference is found in the partial charges on symmetrical atoms, and if the difference is greater than a threshold value, adjusting the partial charges of the corresponding symmetrical atoms.

21. The method of claim 20, wherein said step (iii) comprises:
averaging the corresponding partial charges of the atoms to produce an average partial charge for the atoms, and
replacing each corresponding partial charge with the average partial charge.

22. The method of claim 1, wherein said step (i) comprises:
determining a center point of the selected conformer;
determining the size of the smallest sphere that could encompass the selected conformer while centered at the center point; and
defining a fragment-fragment cutoff value for the selected conformer to be the diameter of the sphere.

23. The method of claim 22, wherein the center point is the center of mass of the selected conformer.

24. The method of claim 22, wherein the center point is the geometric center of the selected conformer.

25. A method of deriving data regarding a fragment molecule that is under evaluation regarding its interaction with a protein, comprising calculating an energy offset for said fragment molecule.
